(19) United States
(12) Patent Application Publication
(10) Pub. No.: US 2015/0250517 A1
(43) Pub. Date: Sep. 10, 2015

(54) BONE CEMENT COMPONENT INJECTION SYSTEM WITH REDUCED FUME EXPOSURE AND METHOD

(71) Applicant: KYPHON SARL, Neuchatel (CH)

(72) Inventor: Neil Sasaki, Santa Clara, CA (US)

(21) Appl. No.: 14/718,332

(22) Filed: May 21, 2015

Related U.S. Application Data
(63) Continuation of application No. 13/446,277, filed on Apr. 13, 2012.

(51) Int. Cl. A61B 17/88

(52) U.S. Cl. CPC A61B 17/8827; A61B 17/8833

(57) ABSTRACT

A system for containing fumes for a bone cement component includes a vial holder configured for receiving and holding a vial therein. A holder chamber is configured to receive and secure the vial holder therein such that when engaged with the vial holder, the vial holder and the holder chamber form an enclosure for containing the vial. A vial-breaking device is disposed in the holder chamber and is configured to break the vial to release contents of the vial into the holder chamber. A port is in communication with the holder chamber through a filter so that when a vacuum is drawn at the port by a dispensing device, the contents of the vial can be drawn through the filter and into the dispensing device and the fumes are contained in the enclosure. Methods of use are disclosed.

### TECHNICAL FIELD

The present disclosure generally relates to medical devices for bone repair, and more particularly to a component injection and mixing system and method for reducing fumes during bone cement preparation.

### BACKGROUND

Many medical procedures employ medical grade cement in connection with the restoration and strengthening of bone structures. During such procedures, cement is typically dispensed to a bone to fill in voids or spaces in the bone or between medical devices or implants attached to or embedded within the bone. These dispensing devices may include systems as simple as syringes and as complex as electronically controlled valves.

Mixing bone cement, such as, e.g., High-Viscosity Radiopaque (HV-R) bone cement, requires integration of two materials, a monomer solution (a liquid that is packaged in a glass vial) and a powder. One difficulty with the cement monomer is that it generates toxic fumes that ideally need to be contained to prevent inhalation exposure by medical staff. Current cement mixing techniques involve breaking the monomer vial and pouring the fluid into the powder by hand, then sealing a mixing chamber. This results in ambient exposure to fumes from the monomer component. This disclosure describes improvements over these prior art technologies.

### SUMMARY

Accordingly, an injection system for mixing bone cement and method are provided. In one embodiment, in accordance with the principles of the present disclosure, a system for containing fumes for a bone cement component is provided. The system comprises a vial holder configured for receiving and holding a vial therein. A holder chamber is configured to receive and secure the vial holder therein such that when engaged with the vial holder, the vial holder and the holder chamber form an enclosure for containing the vial. A vial-breaking device is disposed in the holder chamber. The vial holder is configured to be advanced toward the vial-breaking device for breaking the vial and for releasing contents of the vial into the holder chamber. A filter is disposed within the holder chamber. A port is in communication with the holder chamber through the filter such that when a vacuum is drawn at the port by a dispensing device, the contents of the vial can be drawn through the filter and into the dispensing device such that fumes are contained in the enclosure and the dispensing device.

In one embodiment, the system comprises a vial holder configured for receiving a vial therein and including a holding structure for maintaining the vial in the vial holder. The vial holder has an opening to expose a surface of a vial. The vial includes a monomer component for bone cement. A holder chamber is configured to receive and secure the vial holder. The vial holder is threadedly received in the holder chamber and is advanced in an advance direction by employing the threads. The vial holder and the holder chamber form an enclosure for containing the vial. A vial-breaking device is disposed in the holder chamber. The vial holder is configured to be advanced toward the vial-breaking device for engaging the surface of the vial, breaking the vial and releasing contents of the vial into the holder chamber. A filter is disposed within the holder chamber. A port is in communication with the holder chamber through the filter. A one-way valve is disposed between the holder chamber and the port to prevent back flow into the holder chamber. A dispensing device is connectable to the port for drawing a vacuum at the port to draw the contents of the vial through the filter and into the dispensing device such that fumes are contained in the enclosure and the dispensing device.

In one embodiment, a method for containing fumes of a bone cement component is provided. The method comprises the steps of: providing a system including a vial holder configured for receiving and holding a vial therein; a holder chamber configured to receive and secure the vial holder therein such that when engaged with the vial holder, the vial holder and the holder chamber form an enclosure for containing the vial; a vial breaking device disposed in the holder chamber, the vial holder being configured to be advanced toward the vial breaking device for breaking the vial and for releasing contents of the vial into the holder chamber; a filter disposed within the holder chamber; and a port in communication with the holder chamber through the filter; providing a vial of a monomer component of bone cement in the vial holder; advancing the vial holder in the holder chamber to break the vial against the vial breaking device; and drawing a vacuum at the port by a dispensing device to draw the contents of the vial through the filter and into the dispensing device such that fumes are contained in the enclosure and the dispensing device.

### BRIEF DESCRIPTION OF THE DRAWINGS

The present disclosure will become more readily apparent from the specific description accompanied by the following drawings, in which:

FIG. 1 is a partial cross-sectional view showing a system for extracting liquid from a vial for one embodiment in accordance with the principles of the present disclosure;

FIGS. 2A-2C are perspective views of components of the system shown in FIG. 1 in use for breaking a vial, drawing its contents through a filter and loading the contents into a syringe with minimal exposure to fumes in accordance with the principles of the present disclosure;

FIG. 3 is a cross-sectional view of a mixing chamber having a needle on a syringe passing through a membrane of a sealed enclosure of the mixing chamber to provide minimal exposure to fumes in accordance with the principles of the present disclosure;

FIG. 4 is a partial cross-sectional view showing another system for extracting liquid from a vial for one embodiment in accordance with the principles of the present disclosure;

FIGS. 5A-5C are perspective views of components of the system shown in FIG. 4 in use for breaking a vial, drawing its contents through a filter and loading the contents into a syringe with minimal exposure to fumes in accordance with the principles of the present disclosure;

FIG. 6 is a cross-sectional view of a mixing chamber having a port for connecting with a syringe on a sealed enclosure of the mixing chamber to provide minimal exposure to fumes in accordance with the principles of the present disclosure.

Like reference numerals indicate similar parts throughout the figures.

### DETAILED DESCRIPTION

The exemplary embodiments of an injection system for mixing bone cement and related methods of use are disclosed in terms of medical devices for the treatment skeletal disorders and more particularly, in terms of a medical system and method for mixing bone cement while limiting exposure to fumes. It is envisioned that the medical system and method may be employed in applications such as bone treatment and repair surgeries. For example, the medical system and method can include new mixing chamber configurations.

In particularly useful embodiments, injection and mixing devices are provided that allow for breaking a monomer vial in an enclosed space to prevent fume exposure, and for injecting the monomer into a sealed mixing chamber via a fitting, such as, a Luer-lock or similar fitting. These embodiments utilize a threaded chamber, which advances the monomer vial onto a vial-breaking device, e.g., a metal spike or needle. This spike causes the vial to break, permitting fluid to seep out of the vial. A connected syringe has its plunger drawn, which pulls the monomer through a filter, preventing any glass shards from passing therethrough. The monomer flows through the filter, into a syringe barrel. The device is then attached to a sealed cement-mixing chamber (with powder already in place). Once connected, the user will advance the syringe plunger injecting the monomer. To prevent monomer from flowing back into the vial chamber, a one-way valve is utilized and permits flow into the syringe but not up into the chamber.

In one embodiment, a tool used to open and introduce the monomer to bone cement powder is in a completely sealed system. The tool prevents hazardous fumes from being released and permits for the controlled introduction of monomer to powder. The present embodiments may be employed in conjunction with known cement mixers (e.g., Kyphon™ cement mixers) to introduce monomer into powder in a controlled fashion. The tool in accordance with the present principles may be integrated into cement mixer designs to prevent fume exposure. Hospitals, in particular European hospitals, are becoming increasingly sensitive to fume exposure during mixing of cements. Use of the monomer introduction devices in accordance with the present principles reduce or prevent fume exposure.

It is contemplated that one or all of the components of the medical system may be disposable, peel-pack, pre-packed sterile devices. One or all of the components of the medical system may be reusable. The surgical system may be configured as a kit with multiple sized and configured components.

It is envisioned that the present disclosure may be employed to treat or repair bone injuries or disorders such as, for example, osteoporosis, joint replacement, fracture repairs, bone breaks, etc. It is contemplated that the present disclosure may be employed with other osteal and bone related applications, including those associated with diagnostics and therapeutics. It is further contemplated that the disclosed medical systems and methods may be alternatively employed in a surgical treatment with a patient in a prone or supine position, and/or employ various surgical approaches, including anterior, posterior, posterior mid-line, direct lateral, postero-lateral, antero-lateral approaches, etc. in any body region. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

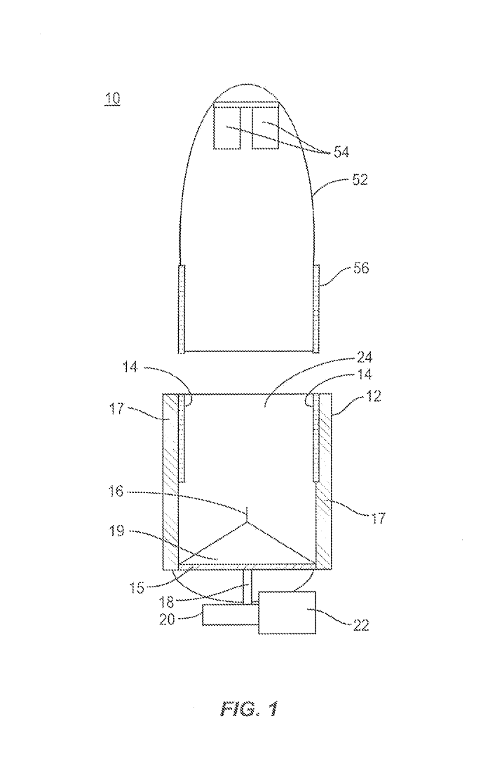

The following discussion includes a description of a mixing system and related methods of employing the system in accordance with the principles of the present disclosure. Alternate embodiments are also disclosed. Reference will now be made in detail to the exemplary embodiments of the present disclosure, which are illustrated in the accompanying figures. Turning now to FIGS. 1-6, there are illustrated components of a medical system, such as, for example, a component extraction system 10 for use with bone cement in accordance with the principles of the present disclosure.

The components of system 10 (or 80) can be fabricated from biologically acceptable materials suitable for medical applications, including metals, synthetic polymers, ceramics, glass and bone material and/or their composites, depending on the particular application and/or preference of a medical practitioner. For example, the components of system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of system 10 may have material composites, including the above materials, to achieve various desired characteristics such as strength, rigidity, elasticity, compliance, biomechanical performance, durability and radiolucency or imaging preference. The components of system 10, individually or collectively, may also be fabricated from a heterogeneous material such as a combination of two or more of the above-described materials. The components of system 10 may be monolithically formed, integrally connected or include fastening elements and/or instruments, as described herein.

System 10 is employed, for example, with an open, mini-open or minimally invasive surgical technique to fill voids, provide patches, attach prosthetic devices, etc., or any other bone related repairs or treatments.

Referring to FIG. 1, an extraction device 10 is illustratively shown in accordance with one embodiment. Device 10 includes a holder chamber 12 configured to receive a vial (not shown), e.g., a vial containing a monomer for activating bone cement. Device 10 may or may not be shipped with a vial contained therein. Holder chamber 12 includes walls 17 that form a cavity 24 into which the vial can be inserted. The cavity 24 may include threads 14 or other mechanism which positions and steadies the vial on a vial-breaking device 16, e.g., a needle or spike 16. A vial holder 52 is configured to receive and contain a vial therein. The vial holder 52 includes a support structure 54 which can hold the vial against gravity when assembling the vial holder 52 on the holder chamber 12. Structure 54 may include elastic pieces or other structures, which are configured to pinch, squeeze or otherwise hold a vial therebetween.

During use, threads 56 of the vial holder 52 are engaged with threads 14 of the holder chamber 12. Once the threads 14 and 56 are engaged the vial is enclosed therein to reduce fumes when the vial is opened. In one embodiment, the vial-breaking device 16 includes a hollow needle to permit fluid flow therethrough. Alternately, the vial-breaking device 16 may be solid (e.g., spike) and permit fluid to flow over and into openings (not shown) in a base 19. In either case, the fluid from the vial would flow onto and cover a filter 15.

Filter 15 may include a porous solid, a fabric or mesh configured to trap particles, or foreign material including glass or fragments from the breakage of the vial. A one-way valve 18 is provided to receive filtered material. The one-way valve 18 ensures that liquid moving therethrough is not permitted to flow back and reenter the cavity 24. A syringe-receiving cavity 20 fluidly communicates with the one-way valve 18. A fitting or port 22 provides a sealed attachment point for a dispensing device or syringe (not shown). The fitting 22 may include a Luer-lock fitting or other fitting. When the dispensing device is docked with the port 22, in this embodiment, the dispensing device is disposed transversely to an advancing direction of the vial holder 52.

In use, a syringe is connected to the fitting 22. The vial is drawn down on the needle or spike 16 to breach the vial to release its contents. The contents flow down to the fitter 15. A plunger of the syringe is moved to draw the contents through the filter 15, the one way valve 18 and into a barrel of the syringe with minimal fumes escaping.

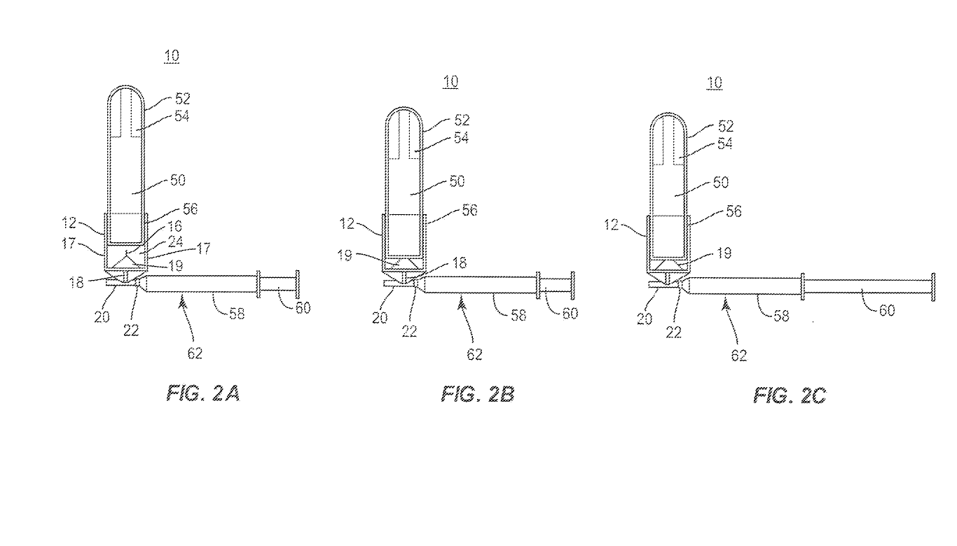

Referring to FIGS. 2A-2C, a method for employing the device 10 of FIG. 1 is illustratively shown in accordance with one embodiment. FIG. 2A includes a vial 50 inside the vial holder 52. The vial holder 52 includes a structure or spacer 54 to support the vial 50 and provide a spacing to enable proper operation of the device 10. The vial holder 52 encloses the vial 50 except at a base of the vial 50, which will remain exposed to engage the needle or spike 16. The vial holder 52 includes threads 56 or other connection mechanism to engage threads 14 on walls 17 of the holder chamber 12. A syringe 62 is attached to the fitting 22. A plunger 60 of the syringe is in a fully advanced (closed) position within a barrel 58 of the syringe 62.

In FIG. 2B, the vial 50 is advanced by screwing down the vial holder 52 using, e.g., threads 56. As the vial 50 is advanced, the vial 50 is broken when it comes in contact with the needle 16 at a base of the holder chamber 12. The broken vial 50 permits the fluid to flow to the filter 15.

In FIG. 2C, the plunger 60 is extended (opened) to draw liquid contents of the vial 50 through the filter 15, through the one-way valve 18 into the syringe chamber or barrel 58. The liquid contents are contained at all times within the vial 50, the holder chamber 12 (and vial holder 52), and, finally, the syringe barrel 58. Since the entire procedure maintains the contents of the vial 50 within the enclosure formed by the syringe 62 and components of the system 10, fumes from the contents of the vial 50 are reduced, minimized or eliminated.

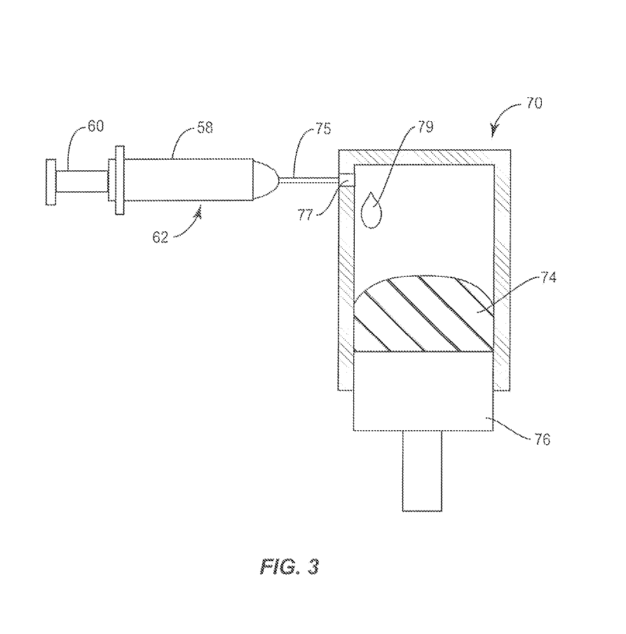

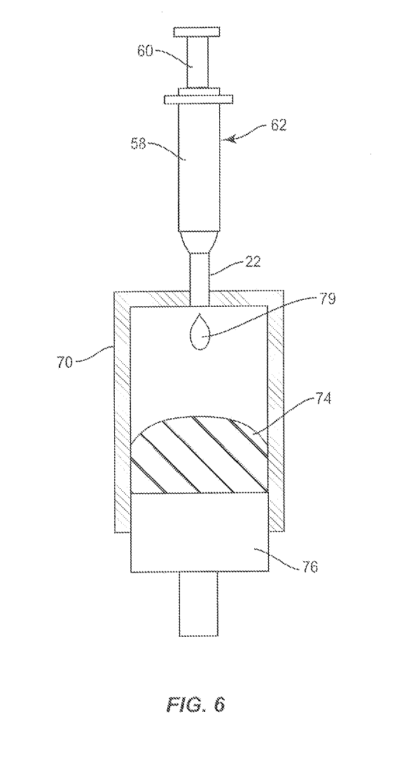

Referring to FIG. 3, the syringe 62 carrying the contents of the vial 50 is transported to an enclosed/sealed-mixing chamber 70. The syringe 62 is fitted with a needle or tip 75 which may be employed to pierce a membrane or other resealing material 77 in the wall of the mixing chamber 70. The contents of the syringe barrel 58 are emptied into the mixing chamber 70 by advancing the plunger 60, which includes a powder 74 for forming bone cement. Since the syringe is enclosed and the mixing chamber is enclosed, ambient fumes are minimized. Other methods for injecting a liquid (e.g., monomer component) 79 into the chamber 90 are also contemplated (See e.g., FIG. 6).

In this example, the bone cement may be formed from a powder 74 (e.g., pre-polymerized PMMA and/or PMMA or MMA co-polymer beads and/or amorphous powder, radio-opacifer, initiator) and the liquid 79 (e.g., MMA monomer, stabilizer, inhibitor). The two components are mixed, and a free radical polymerization occurs when the components are mixed. The bone cement viscosity changes over time from a runny liquid into a dough-like material that can be applied and then finally hardens into solid hardened material. The mixture is mixed or infused by a mixing element 76.

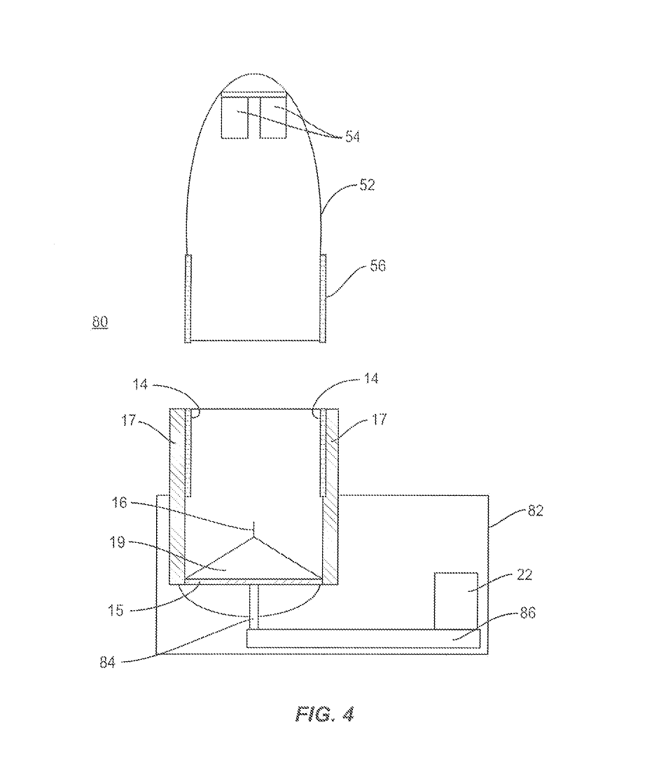

Referring to FIG. 4, another extraction device 80 is illustratively shown in accordance with another embodiment. System 80 is equivalent to system 10 except as noted. Here, the dispersing device or syringe (not shown) is docked in the fitting or port 22 in a parallel orientation relative an advancing direction of the vial holder 52. Device 80 includes a base assembly 82, which includes a vertically disposed holder chamber 12 configured to receive a vial (not shown), e.g., a vial containing a monomer for activating a bone cement. Device 80 may or may not be shipped with a vial contained therein. Holder chamber 12 forms the cavity 24 into which the vial can be inserted. As before, the vial holder 52 is configured to receive and contain a vial therein. The vial holder 52 includes the support structure 54 which can hold the vial against gravity when assembling the vial holder 52 on the holder chamber 12. Structure 54 may include elastic pieces or other structures, which are configured to pinch, squeeze or otherwise hold a vial therebetween.

During use, threads 56 of the vial holder 52 are engaged with threads 14 of the holder chamber 12. Once the threads 14 and 56 are engaged the vial is enclosed therein to reduce fumes when the vial is opened. The vial-breaking device 16 may include a hollow needle or spike.

The contents of the vial may flow into a one-way valve 84 provided to receive filtered material. The one-way valve 84 ensures that liquid moving therethrough is not permitted to flow back and reenter the cavity 24. A lumen or tube 86 stores the liquid released from the vial. The fitting 22 fluidly communicates with the lumen 86. The fitting 22 provides a sealed attachment point for a syringe (not shown). The fitting 22 may include a Luer-lock fitting or other fitting.

In use, a syringe is connected to the fitting 22. The vial is drawn down on the needle or spike 16 to breach the vial to release its contents. The contents flow down to the filter 15. A plunger of the syringe is moved proximally to draw the contents through the filter 15, through the one-way valve 84, through the lumen 86 and into a barrel of the syringe with minimal fumes escaping.

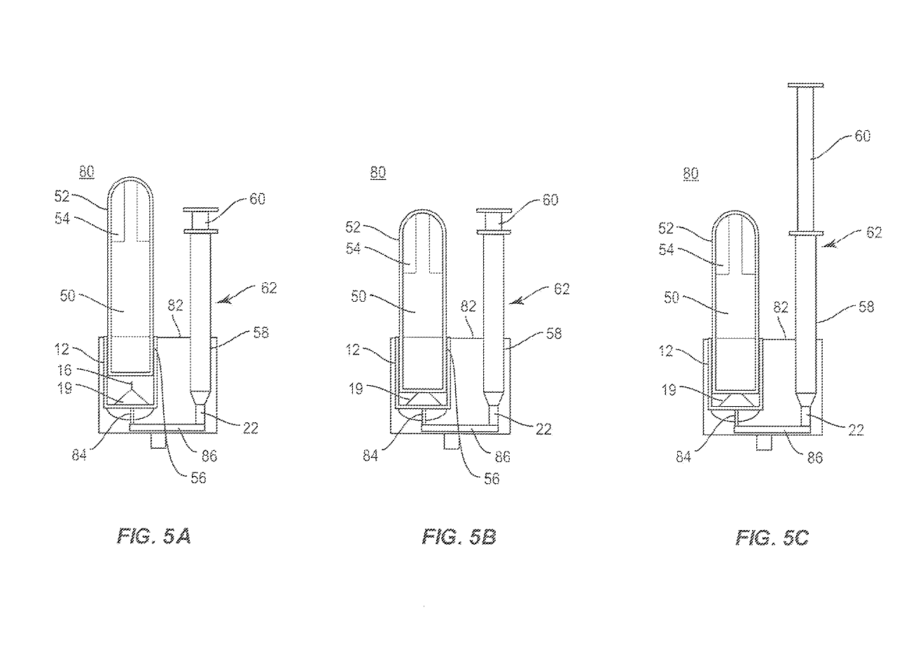

Referring to FIGS. 5A-5C, a method for employing the device 80 of FIG. 4 is illustratively shown in accordance with one embodiment. FIG. 5A includes the vial 50 inside the vial holder 52. The vial holder 52 includes the structure or the spacer 54 to support the vial 50 and provide spacing to enable proper operation of the device 80. The vial holder 52 encloses the vial 50 except at a base of the vial 50, which will remain exposed to engage the needle or spike 16. The vial holder 52 includes threads 56 or other connection mechanism to engage walls 17 of the holder chamber 12. The syringe 62 is attached to the fitting 22. The plunger 60 of the syringe is in a fully advanced (closed) position within the barrel 58 of the syringe 62.

In FIG. 5B, the vial 50 is advanced by screwing down the vial holder 52 using, e.g., threads 56. As the vial 50 is advanced, the vial 50 is broken when it comes in contact with the needle 16 at a base of the holder chamber 12. The broken vial 50 permits the fluid to flow to the filter 15.

In FIG. 5C, the plunger 60 is extended (opened) to draw liquid contents of the vial 50 through the filter 15, through the one-way valve 84, through the lumen 86 and into the syringe chamber or barrel 58. The liquid contents are contained at all times within the vial 50, the holder chamber 12 (and vial holder 52), and, finally, the syringe barrel 58. Since the entire procedure maintains the contents of the vial 50, fumes from the contents of the vial 50 are reduced, minimized or eliminated.

Referring to FIG. 6, the syringe 62 carrying the contents of the vial 50 is transported to the enclosed mixing chamber 70 in the same way as previously described. In this embodiment, the syringe 62 may be attached or injected into the mixing chamber 70 using a fitting 72 instead of the needle 75 (FIG. 3). The contents of the syringe barrel 58 are emptied into the mixing chamber 70 by advancing the plunger 60. The mixing element 76 is employed to mix the two components, e.g., monomer 79 and powder 74 to obtain bone cement for a medical application. Throughout the entire process including mixing the bone cement, the monomer is closed off to the ambient environment to contain the disbursement of noxious fumes before and during a medical procedure. Once the monomer is extracted from the vial, the systems 10 or 80 may be discarded.

In assembly, operation and use, systems 10 and 80, described above, are employed in a surgical procedure, such as, for the treatment or repair of bones. For example, as shown in FIGS. 1-6, systems 10 and 80 can be employed for safer mixing of bone cement components for the treatment and repair of bones, to strengthen or rebuild bones, etc. It is contemplated that one or all of the components of systems 10 and 80 can be delivered or employed as a pre-assembled device or can be assembled in situ. Systems 10 and 80 may be completely or partially revised, removed or replaced. In one embodiment, the holder chamber 12 (and/or vial holder 52) may be shipped having vials 50 contained therein. In other words, a packed kit may include system 10 (or 80) including a vial 50 contained in a vial holder 52 with structure 54 and an optional syringe 62 with a compatible fitting.

For example, as shown in FIGS. 1-6, systems 10 and 80, described above, can be employed during a surgical procedure for mixing and dispensing bone cement. In use, a medical practitioner obtains access to a surgical site including a bone in any appropriate manner, such as through incision and retraction of tissues. It is envisioned that system 10 or 80 can be used in any existing surgical method or technique including open surgery, mini-open surgery, minimally invasive surgery and percutaneous surgical implantation, whereby the bone is accessed through a mini-incision, or sleeve that provides a protected passageway to the area. Once access to the surgical site is obtained, the particular surgical procedure can be performed for treating or repairing the bone.

An incision is made in the body of a patient and a cutting instrument (not shown) creates a surgical pathway for implantation of components for supplying bone cement mixed using systems 10 or 80. A preparation instrument (not shown) can be employed to prepare tissue surfaces of the bone, as well as for aspiration and irrigation of a surgical region according to the requirements of a particular surgical application.

Holes, fractures, voids, depressions, etc. may exist in the bone or may be created in the bone as part of the procedure. After appropriate steps are taken for the treatment or repair, these holes, fractures, voids, depressions, etc. are filled with the mixed bone cement to maintain or improve the bone's structural integrity. Components are delivered to the surgical site along the surgical pathway(s) and into or onto bone tissue.

In one embodiment, an agent may be mixed with or delivered with bone cement (in, e.g., in the two components) or delivered separately. It is envisioned that the agent may include bone growth promoting material.

It is contemplated that the agent may include therapeutic polynucleotides or polypeptides. It is further contemplated that the agent may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as HA, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines. Components can be made of radiolucent materials such as polymers. Radiomarkers may be included for identification under x-ray, fluoroscopy, CT or other imaging techniques. It is envisioned that the agent may include one or a plurality of therapeutic agents and/or pharmacological agents for release, including sustained release, to treat, for example, pain, inflammation and degeneration.

It is envisioned that the use of microsurgical and image guided technologies may be employed to access, view and repair bone deterioration or damage. Upon completion of the procedure, the surgical instruments and assemblies are removed and the incision is closed.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

1-18. (canceled)

19. A method for containing fumes of a bone cement component, the method comprising the steps of:
providing a system including a vial holder configured for receiving and holding a vial therein; a holder chamber configured to receive and secure the vial holder therein such that when engaged with the vial holder, the vial holder and the holder chamber form an enclosure for containing the vial; a vial breaking device disposed in the holder chamber, the vial holder being configured to advance toward the vial breaking device for breaking the vial and for releasing contents of the vial into the holder chamber; a filter disposed within the holder chamber; and a port in communication with the holder chamber through the filter;
providing a vial of a monomer component of bone cement in the vial holder;
advancing the vial holder in the holder chamber to break the vial against the vial breaking device; and
drawing a vacuum at the port by a dispensing device to draw the contents of the vial through the filter and into the dispensing device such that fumes are contained in the enclosure and the dispensing device.

20. The method for containing fumes of a bone cement component as recited in claim 19, further comprising:
injecting the monomer component into a sealed mixing chamber with a powder component of the bone cement to further contain the fumes and to mix the bone cement.

21. The method for containing fumes of a bone cement component as recited in claim 19, wherein the enclosure and the dispensing device form a completely sealed assembly.

22. The method for containing fumes of a bone cement component as recited in claim 19, wherein drawing the vacuum at the port comprises moving a plunger of the dispensing device that is disposed within a passageway of the dispensing device such that the contents of the vial are drawn into the passageway.

23. The method for containing fumes of a bone cement component as recited in claim 19, wherein:
the vial holder defines a longitudinal axis; and
drawing the vacuum at the port comprises moving a plunger of the dispensing device that is disposed within a passageway of the dispensing device along an axis that is parallel to the longitudinal axis and offset from the longitudinal axis such that the contents of the vial are drawn into the passageway.

24. The method for containing fumes of a bone cement component as recited in claim 19, wherein:
the vial holder defines a longitudinal axis; and
drawing the vacuum at the port comprises moving a plunger of the dispensing device that is disposed within a passageway of the dispensing device along an axis that extends transverse to the longitudinal axis such that the contents of the vial are drawn into the passageway.

25. The method for containing fumes of a bone cement component as recited in claim 19, wherein advancing the vial holder in the holder chamber comprises engaging threads of the vial holder with threads of the holder chamber and rotating the vial holder relative to the holder chamber.

26. The method for containing fumes of a bone cement component as recited in claim 19, wherein the port provides a sealed attachment point for the dispensing device.

27. The method for containing fumes of a bone cement component as recited in claim 19, wherein the port comprises a Luer-lock fitting and the dispensing device is a syringe, and the method further comprises connecting the syringe to the Luer-lock fitting.

28. The method for containing fumes of a bone cement component as recited in claim 19, wherein the port comprises a sealable membrane and the dispensing device includes a needle injectable through the membrane.

29. The method for containing fumes of a bone cement component as recited in claim 19, wherein the system comprises a one way valve disposed between the holder chamber and the port to prevent back flow into the holder chamber.

30. The method for containing fumes of a bone cement component as recited in claim 19, wherein the vial breaking device is a hollow needle configured to permit fluid flow therethrough.

31. A method for containing fumes of a bone cement component, the method comprising:
inserting a vial into a vial holder;
positioning the vial holder within a holder chamber;
moving the vial holder relative to the holder chamber such that the vial is breached by a vial-breaking device in the holder chamber and contents of the vial are released from the vial into an enclosure formed by the vial holder and the holder chamber;
coupling a dispensing device to a port that is in communication with the enclosure; and
drawing a vacuum using the dispensing device to move the contents of the vial from the enclosure and into the dispensing device.

32. The method recited in claim 31, further comprising:
uncoupling the dispensing device from the port;
coupling the dispensing device to a sealed mixing chamber; and
injecting the contents of the vial into the mixing chamber.

33. The method recited in claim 31, wherein the enclosure and the dispensing device form a completely sealed assembly.

34. The method recited in claim 31, wherein drawing the vacuum comprises moving a plunger of the dispensing device that is disposed within a passageway of the dispensing device such that the contents of the vial are drawn into the passageway.

35. The method recited in claim 31, wherein:
the vial holder defines a longitudinal axis; and
drawing the vacuum comprises moving a plunger of the dispensing device that is disposed within a passageway of the dispensing device along an axis that is parallel to the longitudinal axis and offset from the longitudinal axis such that the contents of the vial are drawn into the passageway.

36. The method recited in claim 31, wherein:
the vial holder defines a longitudinal axis; and
drawing the vacuum comprises moving a plunger of the dispensing device that is disposed within a passageway of the dispensing device along an axis that extends transverse to the longitudinal axis such that the contents of the vial are drawn into the passageway.

37. The method recited in claim 31, wherein the
moving the vial holder relative to the holder chamber comprises engaging threads of the vial holder with threads of the holder chamber and rotating the vial holder relative to the holder chamber

38. A method for containing fumes of a bone cement component, the method comprising:
inserting a vial into a vial holder, the vial containing a bone cement monomer;
positioning the vial holder within a holder chamber;
moving the vial holder relative to the holder chamber such that the vial is breached by a vial-breaking device in the holder chamber and the monomer is released from the vial into an enclosure formed by the vial holder and the holder chamber;
coupling a dispensing device to a port that is in communication with the enclosure;
drawing a vacuum using the dispensing device to move the monomer from the enclosure, through a filter in the holder chamber and into the dispensing device;
uncoupling the dispensing device from the port;
coupling the dispensing device to a sealed mixing chamber;
injecting the monomer into the mixing chamber that contains bone cement powder; and
mixing the monomer with the powder using a mixing element of the mixing chamber.
